(19) United States
(12) Patent Application Publication
(10) Pub. No.: US 2015/0164772 A1
(43) Pub. Date: Jun. 18, 2015

(54) Methods and Compositions to Impart Memory Effects onto Biosurfaces

(71) Applicant: Avon Products, Inc., Suffern, NY (US)

(72) Inventors: Allwyn Colaco, Morristown, NJ (US); Sen Yang, Highland, NY (US); Ashley L. Howell, Oakland, NJ (US)

(21) Appl. No.: 14/627,600

(22) Filed: Feb. 20, 2015

Related U.S. Application Data
(62) Division of application No. 13/711,736, filed on Dec. 12, 2012, now abandoned.
(60) Provisional application No. 61/569,855, filed on Dec. 13, 2011.

(51) Int. Cl. A61K 8/87; A45D 7/06; A61Q 5/06

(52) U.S. Cl. CPC A61K 8/87; A61Q 5/06; A45D 7/06; A61K 2800/81

(57) ABSTRACT

This invention relates to methods and compositions for imparting memory effects onto biosurfaces, such as hair, by using shape memory polymers. This invention also relates to kits containing shape memory polymers that are useful for styling hair. The methods, compositions, and kits of the invention permit a user to recover an original hairstyle after it has been disheveled by exposing the hair to an external stimulus, such as heat.

### RELATED APPLICATIONS

This application claims priority to U.S. Provisional Patent Application Ser. No. 61/569,855, filed Dec. 13, 2011, and is a divisional of, and claims priority to, U.S. patent application Ser. No. 13/711,736, filed on Dec. 12, 2012. The contents of each of the two aforementioned applications are hereby incorporated by reference in their entirety.

### FIELD OF THE INVENTION

This invention relates to methods and compositions for imparting memory effects onto biosurfaces, such as hair, by using shape memory polymers. This invention also relates to kits containing shape memory polymers that are useful for styling hair.

### BACKGROUND OF THE INVENTION

Consumers often wish to change hair styles to suit particular situations or events. For instance, at times people with straight hair may want curly hair. Hair may be straightened using a variety of methods, including curlers, curling irons, or permanent wave treatments. The resulting curls, however, have a tendency to relax over time, due to factors such as gravity or application of physical stress to the hair (e.g., further combing, wearing a hat, or sleeping). To maintain curls, people may use styling products to hold their hair in place. Such styling products provide desired effects, but the benefits often are short lived and lost under external influences. For instance, curled hair treated with such styling products may still lose curliness due to wind or contact with water.

At the other extreme, people with curly hair occasionally may want straight hair. Hair may be straightened using a variety of methods, including flat irons, blow drying, chemical straightening, or thermal reconditioning. To keep hair straight, styling products may be applied, including straightening balm or silicone serum. Nonetheless, the desired effects of these products are also often short lived and may be lost due to high humidity or direct contact with water.

When a hairstyle becomes disheveled as a result of external influences (e.g., wind or rain), one typically has to go through the entire styling process again to recover the original hairstyle, which may be time consuming and expensive. Currently, hair styling products do not exist that allow the consumer to revert to an earlier hairstyle with a high degree of recovery.

### SUMMARY OF THE INVENTION

One aspect of this invention is to provide a method for styling keratin fibers, a non-limiting example of which includes hair. The method includes the steps of depositing a film of a thermoplastic or thermosetting shape memory polymer (SMP) on a length of the keratin fibers. The SMP preferably has a transition temperature (Tₜᵣₐₙₛ) in the range of about 120 to about 180° F. (about 49 to about 82° C.). The SMP preferably is capable of forming chemical or physical cross-links at a temperature in the range of about 300 to about 400° F. (about 149 to about 204° C.). The method includes styling the keratin fibers at a temperature in the range of about 300 to about 400° F. (about 149 to about 204° C.) to cause the SMP to form such cross-links.

Another aspect of the invention is to provide a method for recovering a desired hairstyle of a subject. The method includes applying a shape memory polymer to the hair of a subject. The shape memory polymer comprises a hard segment and a soft segment. The soft segment has a transition temperature Tₜᵣₐₙₛ above which the soft segment melts or undergoes a glass transition. The method further includes the steps of styling the hair to a desired hairstyle and setting the desired hairstyle by heating the hair to a setting temperature Tₛₑₜ that is greater than or equal to Tₜᵣₐₙₛ. The method also includes the step of subsequently cooling the hair to a temperature below Tₜᵣₐₙₛ. The method allows one recovering the desired hairstyle whenever it is lost by reheating the hair to a temperature greater than or equal to Tₜᵣₐₙₛ.

Another aspect of the invention provides a composition for styling hair. The composition comprises a shape memory polymer that is present in an effective amount for styling hair. The composition optionally may also contain a cosmetically acceptable solvent and a viscosity-modifying agent. In certain embodiments where the composition contains a viscosity-modifying agent, it is preferable that the amount of viscosity-modifying agent present in the composition is sufficient to give the composition a viscosity in the range of 1000 to 100,000 cP at 20° C.

Yet another aspect of the invention is to provide a kit for styling hair. In certain preferred embodiments, the kit includes a first compartment containing a composition comprising a thermoset resin and a second compartment containing a composition comprising a hardener. The thermoset resin and hardener are capable of forming a shape memory polymer when the compositions of the first and second compartments are mixed to form a hair styling composition. Preferably, at least one of the compositions in the first or second compartment further contains a cosmetically acceptable solvent. It is also preferable that at least one of the compositions in the first or second compartments contains a viscosity-modifying agent that causes the hair styling composition to have a viscosity in the range of 1000 to 100,000 cP at 20° C.

### DETAILED DESCRIPTION OF THE INVENTION

A shape memory polymer is a polymer that has the ability to “remember” an initial shape and to recover the initial shape after the polymer has been deformed. Typically, the recovery of the initial shape after deformation is induced by an external stimulus, non-limiting examples of which include heat, pH, electric current, UV-radiation, visible light, infrared radiation, near-infrared radiation and microwave radiation. Shape memory polymers have been described previously. See, e.g., Lendlein, A. et al. “Shape Memory Polymers,” Angew. Chem. Int. Ed. 2002 41, 2034-2057 (2002); Lendlein, A. et al., “Shape Memory Polymers,” Kirk Othmer Encyclopedia of Chemical Technology, (5^{th} Ed.) Vol. 22, p. 355-365 (2006). The contents of these (and all other cited references herein) are expressly incorporated by reference.

The unusual ability of a shape memory polymer to “remember” an initial shape arises from the structure and morphology of the shape memory polymer. Without wishing to be bound by theory, it is believed that shape memory polymers contain at least two separated phases. The phase that has the highest melting point—typically referred to as the “hard segment”—is responsible for the permanent shape of a given shape memory polymer. The shape memory polymer can be processed into a “permanent” shape that is “remembered” by the polymer by heating the shape memory polymer to at least the melting temperature (Tₚₑᵣₘ) of the hard segment, shaping the polymer, and then allowing it to cool below Tₚₑᵣₘ. Furthermore, and again without wishing to be bound by theory, it is believed that shape memory polymers contain at least a second phase, typically referred to as either a “soft segment” (or “switching segment”). It is believed that the soft segment acts as a “molecular switch” that allows the shape memory polymer to assume a temporary shape and to recover its initial (permanent) shape upon application of an external stimulus.

By way of example, for thermally responsive shape memory polymers, it is believed that the soft segment converts from a rigid, plastic state to a flexible, elastic state upon heating the shape memory polymer above a certain transition temperature, Tₜᵣₐₙₛ (where Tₜᵣₐₙₛ<Tₚₑᵣₘ). Depending on the specific shape memory polymer system, Tₜᵣₐₙₛ may be either the glass-transition temperature (T_{g}) or the melting temperature (Tₘ) of the soft segment. Thus, one can cause a thermally responsive shape memory polymer to assume a temporary shape by heating the shape memory polymer above Tₜᵣₐₙₛ, shaping the polymer, and allowing it to cool to a temperature below Tₜᵣₐₙₛ. To recover the original (“permanent”) shape again, the shape memory polymer is simply heated again to a temperature above Tₜᵣₐₙₛ.

Without wishing to be bound by theory, it is believed that shape memory polymers at a molecular level consist of networks of polymeric chains that are connected at regions known as netpoints. In some shape memory polymers, the netpoints are formed by entanglement of the polymeric chains or by intermolecular interaction of certain polymer blocks within the shape memory polymer. When netpoints form in this manner, the shape memory polymer is said to be physically cross-linked. Netpoints also can form as the result of covalent bonding. When a shape memory polymer contains netpoints arising from covalent bonds, the shape memory polymer is said to be chemically cross-linked. Chemically cross-linked shape memory polymers are also referred to as thermoset or semi-crystalline network shape memory polymers.

One aspect of this invention is to use shape memory polymers in a method for styling keratin fibers (e.g., hair). The method includes a step of depositing a film of a shape memory polymer to the keratin fibers, styling the keratin fibers, and then setting the style by heating the shape memory polymer to Tₚₑᵣₘ or higher. The shape memory polymers contemplated by this aspect of the invention are not particularly limited, and include any polymer possessing shape memory characteristics with a Tₚₑᵣₘ and a Tₜᵣₐₙₛ in a temperature range that permits styling without causing excessive thermal damage to the keratin fiber. When the keratin fiber is hair, the Tₚₑᵣₘ and Tₜᵣₐₙₛ of the shape memory polymers preferably are in a temperature range that can be achieved using conventional hair styling devices, non-limiting examples of which include hair dryers, curling irons, curler dryers, hot curlers, straightening irons, and the like. In this regard, useful shape memory polymers include those that have a Tₚₑᵣₘ in the temperature range with a lower limit of 120, 125, 130, 135, 140, 145, 150, 155, 160, 165 or 170° C. and an upper limit of 175, 180, 185, 190, 195, 200, 205, 210, 215, 220, 225, 230, or 240° C., with each combination of upper and lower temperature values being an expressly contemplated embodiment of the invention. In addition, suitable shape memory polymers include those with a Tₜᵣₐₙₛ temperature range having a lower temperature limit of 40, 45, 50 , 55, 60, 65 or 70° C. and an upper temperature limit of 75, 80, 85, 90, 95 or 100° C., with each combination of upper and lower temperature values being an expressly contemplated embodiment of the invention.

Shape memory polymers are commercially available. Non-limiting examples of shape memory polymers include those in the group consisting of epoxy thermoset polymers (e.g., TEMBO®; Composite Technology Development, Lafayette, Colo.). Other commercially available shape memory polymers include polynorbornene (i.e., NORSOREX®, Astrotech, Austin, Tex.), aliphatic polyether-based thermoplastic polyurethanes (i.e., TECOFLEX®, Lubrizol, Wickliffe, Ohio), thermoplastic polyurethanes (i.e., TECOPHILIC®, Lubrizol, Wickliffe, Ohio), aliphatic, polycarbonate-based thermoplastic polyurethanes (i.e., CARBOTHANE®, Lubrizol, Wickliffe, Ohio), and aromatic polyether-based thermoplastic polyurethanes (i.e., TECOTHANE®, Lubrizol, Wickliffe, Ohio), and polyaryletheretherketone (i.e., PEEK ALTERA®, Medshape Solutions, Atlanta, Ga.), and combinations thereof.

Shape memory polymers can be synthesized through free-radical polymerization to achieve Tₘ's and T_{g}'s of interest. For example, ureidopyrimidinone substituted ethyl methacrylate monomer (Upy-EMA), butyl acrylate (BA), trimethylolpropane trimethacrylate, 2,2′-Azobis(2-methylpropionitrile), AIBN may be combined in 1-methyl-2-pyrrolidinone at a mol ratio of BA: TMP-TMA: UPy-EMA : AIBN of 96.5:1.5:2:0.5. This polymer network with reversibly associated side-groups resulted in a T_{g} of −66° C. As the skilled artisan will appreciate, Tₜᵣₐₙₛ of such polymers can be adjusted by varying the type, molecular weight, and weight fraction of the monomers, crosslinking agents, and initiators to result in Tₜᵣₐₙₛ values in the 40-50° C. range. Examples of acrylate and methacrylate monomers include, but are not limited to, PEG-22 dimethacrylate, triethylene glycol dimethacrylate, tetraethylene glycol dimethacrylate, 1,12-dodecanediol dimethacrylate, urethane dimethacrylate, diurethane dimethacrylate, trimethylhexyl dicarbamate, poly(ethyleneglycol) (400) dimethacrylate,: PEG-22 dimethacrylate, trimethylolpropane trimethacrylate, ethoxylated bis phenol A dimethacrylate (EO=6, 8, 10, 30), PEG 400 extended urethane dimethacrylate, trimethylhexyl dicarbamate, isophorone urethane dimethacrylate (UDMA-IPDI), Di-HEMA trimethylhexyl dicarbamate, butyl acrylate, tert-butyl acrylate, hydroxypropyl methacrylate (HPMA), hydroxypropyl methacrylate, ethayl methacrylate, butyl methacrylate, isobutyl methacrylate, 2-ethylhexyl methacrylate, and ureidopyrimidinone substituted ethyl methacrylate monomer (Upy-EMA). The dimethyacrylate reactants listed above are commercially available from Esstech, Inc. (Essington, Pa.). Examples of photoinitiators include, but are not limited to, 1-hydroxy-cyclohexylphenyl-ketone, 2-hydroxy-2-methyl-1-phenyl-1-propanone, diphenyl (2,4,6-trimethylbenzoyl)-phosphine oxide, and 2,2′-azobis(2-methylpropionitrile), which are available from Ciba Specialty Chemicals under the tradenames of IRGACURE®, DAROCUR® and LUCERIN®.

Another interesting class of shape memory polymers are polyurethane-based shape memory polymers. Included in this class of shape memory polymers are polymers with hard segments, e.g., diisocyanates and small size diols or diamines, and soft segments, e.g. aliphatic polyethers or polyesters or hydroxyls. As the skilled artisan will appreciate, Tₜᵣₐₙₛ of such polymers can be adjusted by varying the weight fraction and molecular weight of the hard and soft segments, with Tₜᵣₐₙₛ values in the 40-50° C. range achievable. See, e.g., Lendlein, A. et al., “Shape Memory Polymers,” Kirk Othmer Encyclopedia of Chemical Technology, (5^{th} Ed.) Vol. 22, p. 362 (2006). Examples of soft segments include, but are not limited to, polyester diol, polycarbonate diol, poly(L-lactide), PEG-180 bispolylactide, poly(L-lactide) diol, polylactide-block-poly(ethylene glycol)-block-polylactide, polyglycolide, poly(L-lactide-co-caprolactone-co-glycolide), poly(D,L-lactide-co-glycolide), poly(D,L-lactide-co-caprolactone), poly(hexamethylene carbonate) diol, poly(ethylene-co-1,2-butylene)diol, poly(tetrafluoroethylene oxide-co-difluoromethylene oxide) α,ω-diol, polycaprolactone, poly(caprolactone) diols, and poly(lactide) triols. Examples of hard segments include, but are not limited to, 1,6-hexamethylenediisocyanate, 3,3′-dimethoxy-4,4′-biphenylene diisocyanate, 1,4-phenylene diisocyanate, 4,4′-MDI, and 4,4′-methylenebis(phenyl isocyanate). Chain extenders are frequently added to tune the physical and chemical properties of polyurethanes. Examples include, but are not limited to, ethylene glycol, 1,4-butanediol, hexanediol, and trimethylolpropane.

As explained in Lendlein, A. et al., “Shape Memory Polymers,” Kirk Othmer Encyclopedia of Chemical Technology, (5^{th} Ed.) Vol. 22, p. 362 (2006) and Lendlein, A. et al., “Shape Memory Polymers,” Kirk Othmer Encyclopedia of Chemical Technology, (5^{th} Ed.) Vol. 22, p. 355- 365 (2006), polyurethane based shaped memory polymers may be synthesized using isocyanate-terminated pre-polymers obtained by reacting an excess of low molecular weight diisocyantes with difunctional, hydroxyl-terminated oligoesters and/or oligoethers, with the addition of low molecular weight diols and/or diamines as chain extenders. The polymers so-produced may be, for example, linear, phase-segregated polyurethane block copolymers or polyurethane-urea block copolymers. The transition temperature Tₜᵣₐₙₛ of these polymers may be either a melting temperature Tₘ or a glass transition temperature T_{g}. Shape memory can be affected using polymers with Tₜᵣₐₙₛ=Tₘ where Tₘ is the melting point of the hard segment and the polymer is heated above the crystalling melting point. Examples of this type of shape memory polymer include polyurethanes, polyurethanes with ionic or mesogenic components, block copolymers consisting of polyethyleneterephthalate (PET) and polyethyleneoxide (PEO), block copolymers containing polystyrene and poly(1,4-butadiene), and ABA triblock copolymer made from poly(2-methyl-2-oxazoline) (A block) and polytetrahydrofuran) (B block). Such polymers may, for example, comprise hard-segments composed of MDI/1,4-butanediol; MDI/1,4-butanediol dimethyloylpropionic acid; MDI/BEBP or BHBP; HDI/4,4′-dihyroxybiphenyl; poly(ethylene terephthalate; polystyrene; poly(2-methyl-2-oxazoline); MDI/1,4-butanediol; 2,4-TDI or MDI; carbodiimide-modified diisocyanates (MDI and HDI); ethylene glycol or bis(2-hydroxyethyl)hydrochinone; and a combination of (2,2′-bis(4-hydroxyphenyl)-propane (bis-phenol A) and ethylene oxide. The switching-segment phase may comprise poly(ε-caprolactone) (e.g., with a MW from about 1000 to about 8000); poly(ethylene oxide) (e.g., with a MW of about 400 to 10,000); poly(butadiene). Ionic or mesogenic moieties can be introduced into the hard-segment forming phase to influence the shape-memory properties. For example, 2,2-bis(hydroxymethyl) propionic acid can be added in addition to, or in partial replacement of, the chain extender (e.g., 1,4-butanediol). These hard segments can be combined with any of the soft, switching segment-forming components described herein, including poly(ε-caprolactone) diols. The acid can be neutralized, for example with triethylamine to produce “ionomers” having polyelectrolyte character and hard-segments stabilized by ionic interactions. Mesogenic diols, such as 4,4′-bis(2-hydroxyehtoxy)biphenyl (BEBP) or 4,4′-bis-(2-hydroxyhexoxy)biphenyl (BHBP), can be incorporated into the hard segment (e.g., MDI based) to increase the solubility in the switching block. Shape memory can be affected using polymers with Tₜᵣₐₙₛ=T_{g} where T_{g} is either a glass transition temperature of a pure switching block or a mixed glass transition of the hard and switching phase together. An example of such polyurethane polymers include those with a hard segment of MDI/1,4-butandiol and switching segments such as polyethers (e.g., poly(tetrahydrofuran) and/or polyesters (e.g., poly(ethylene adipate). All other shape memory polymers, including components for the hard segments and switching segments, disclosed in Lendlein, A. et al., “Shape Memory Polymers,” Kirk Othmer Encyclopedia of Chemical Technology, (^{5th} Ed.) Vol. 22, p. 362 (2006) and Lendlein, A. et al., “Shape Memory Polymers,” Kirk Othmer Encyclopedia of Chemical Technology, (5^{th} Ed.) Vol. 22, p. 355- 365 (2006), are hereby incorporated by reference.

Polyurethane-based shape memory polymers with other soft segments are also available. One particularly interesting example is the class of shape memory polymers with a polyurethane hard segment and a poly(tetrahydrofuran) soft segment. Such shape memory polymers can display remarkable shape recovery, with a constant shape recovery of greater than 95% possible even after being deformed˜100 times. See e.g., Lendlein, A. et al., “Shape Memory Polymers,” Kirk Othmer Encyclopedia of Chemical Technology, (5^{th} Ed.) Vol. 22, p. 363 (2006).

Polycaprolactone based shape memory polyurethanes have been synthesized with poly(caprolactone) diol, 1,4-butanediol, 4,4′-MDI, 4,4′-methylenebis(phenyl isocyanate) in “N,N-dimethylformamide. The shape memory polyurethane had a Tₚₑᵣₘ of 200° C. and a T_{g} of 70° C. See e.g., Qinghao Meng, “Polycaprolactone-Based Shape Memory Segmented Polyurethane Fiber” J. Appl. Polymer Sci., Vol. 106, 2515-2523 (2007), Wiley Periodicals, Inc.

Polynorbornene-based SMPs have been synthesized with varying levels of cis- versus trans-norbornene with glass transition temperatures between 35-45° C. Versions that contained polyhedral, oligomeric silsesquioxanes (POSS) to increase the glass transition temperature for improved heat and oxidation resistance have also been synthesized. See, e.g., Angew. Chem. Int. Ed. 2002, 41: 2034-2057.

Other interesting classes of shape memory polymers include natural polymers and natural polymers with synthetic domains. Some non-limiting examples include poly(ethylene glycol)-based SMPs with PEG-α-cyclodextran. See, e.g., Polymer, 49: 3205-3210. SMPs have also been produced that contain peptides.

Properties of the shape memory polymers may be enhanced with inclusion of fillers to the polymeric matrix. Some non-limiting examples of fillers include inorganic fillers such as glass fibers or carbon nanotubes for increasing thermal conductivity, interpenetrating polymers such as poly(ethylene glycol) or inclusion of natural materials such as clay or nanocellulose for improving mechanical properties. See “Shape Memory Polymer Research”Annu. Rev. Mater. Res. 2009, 39:445-471 and Macromol. Rapid Commun., 2006, 27:1100-1104.

In accordance with the principles of the invention, once the shape memory polymer has been applied to the hair, the hair is styled. Generally speaking, the methods and compositions of the invention are suitable for any hairstyle, including curly hairstyles, straight hairstyles, and hairstyles that have both straight and curly components. The styling may be achieved using any known method of styling, non-limiting examples of which include combing, brushing, scrunching, curling, straightening, as well as combinations thereof. Once the styling has achieved the desired look, the hair is heated to at least Tₚₑᵣₘ in order to set the style. In preferred embodiments, heating to Tₚₑᵣₘ is achieved using conventional hair styling devices, including hair dryers, curling irons, curler dryers, hot curlers, straightening irons, and the like. The time for setting the desired hairstyle will be determined according to the individual characteristics of the hair and the style, but may be from about 15 seconds to about one hour, in particular for about 30 seconds.

Typically, after the hairstyle of a subject is set, the hair is allowed to cool to ambient temperature. The subject may then proceed with normal daily activities, some of which invariably will cause changes in the hairstyle. For instance, the subject may expose his or her hair to high humidity, wind, water, and/or physical stress (e.g., combing, wearing a hat, tying up one's hair, or even simply sleeping), all of which may change the hairstyle. In accordance with the principles of the present invention, when the subject wishes to recover the original hairstyle, the subject only needs to heat the hair to a temperature above Tₜᵣₐₙₛ. Preferably, the original hairstyle is restored by convectively heating the hair using heated air, such as from a hair dryer or a curler dryer. However, generally speaking, any method of heating the hair to above Tₜᵣₐₙₛ to recover the original hairstyle is contemplated by the invention, including even heating it above Tₜᵣₐₙₛ by exposing it to warm or hot water.

Preferably, the shape memory polymer is chosen so that the step of recovering the hairstyle involves heating the hair to above Tₜᵣₐₙₛ for a time that is less than the time that was required to achieve the original hairstyle. For example, the step of heating the hair to recover the hairstyle may be performed within a time interval that has a lower limit of about 10, 15, 20 , 25, 30, 35, 40 45, or 50 seconds, and an upper limit of about 60, 70, 80, 90, 100, 110, 120, 130, 140, or 150 seconds, or about one, two, three, four, or five minutes, with each combination of upper and lower limit being an expressly contemplated embodiment of the invention.

In preferred embodiments, the chosen shape memory polymer provides a high amount of curl recovery. The degree to which curliness in the hair can be recovered using shape memory polymers can be measured by a parameter called the “percent curl retention” (also referred to as “percent curl recovery”) which is defined according to the following equation:

$$\% Curl Retention = \frac{{L}_{o} - {L}_{t}}{{L}_{o} - {L}_{p}} \times 100.$$

Here, Lₚ refers to the initial length of the curled hair after at least one curl has been set in the hair by applying a shape memory polymer, heating to a temperature equal to or above Tₚₑᵣₘ, and cooling the hair to ambient temperature. Lₒ refers to the length of the hair after the hair has been set into a temporary shape. Lₜ refers to the length of hair after an external stimulus (e.g., heating) is applied to the hair to recover the original curls. As the skilled artisan will appreciate, if the hair after recovery of the curls has the same length as the original hair (i.e., Lₜ=Lₚ) then the term (L0−Lₜ)/(Lₒ−Lₚ) equals unity and the percent curl retention is 100%. However, in cases where the curls are not completely recovered after application of the external stimulus, then (Lₒ−Lₜ) is less than (Lₒ−Lₚ) and the percent curl recovery is less than 100%. A method for measuring percent curl retention for a given shape memory polymer is discussed in the examples described below.

Another aspect of the invention is to provide a composition comprising a shape memory polymer that is present in an effective amount for styling hair. By “effective amount for styling hair,” it is meant that the shape memory polymer is present in an amount that allows the hair to exhibit shape memory effects after the composition is applied and set as described herein. Preferably, the shape memory polymer is present in an amount sufficient to provide a percent curl retention of at least 50%, 60%, 70%, 80%, 90%, or 95%, as determined using the testing methods and compositions described herein. As the skilled artisan will appreciate, the concentration of shape memory polymer required to achieve a given percent curl retention will be dependent on the chosen shape memory polymer system added to the composition and can be readily determined by routine experimentation for a given shape memory polymer. With this in mind, compositions with some useful concentrations of shape memory polymer include those having a concentration of shape memory polymer in a range having a lower limit of 10, 15, 20, 25, 30, 35, 40, 45, 50 and 55 wt. % and an upper limit of 60, 65, 70, 75, 80, 85, 90, and 95 wt. %, with each combination of upper and lower limit set forth above being an embodiment expressly contemplated by the invention.

Optionally, the shape memory polymer compositions of the invention may contain a viscosity-modifying agent. The presence of a viscosity-modifying agent that increases the viscosity of the composition may be advantageous in certain situations in order to prevent the composition from running while it is being applied to the hair. Preferably, a sufficient amount of viscosity-modifying agent is added to the composition to give the overall composition a viscosity in the range of 1000 to 100,000 cP, or 2000 to 90,000 cP, or 3000 to 80,000 cP, or 4000 to 70,000 cP or 5000 to 60,000 cP at a temperature of 20° C. Generally, any cosmetically acceptable viscosity-modifying agent that does not undesirably react with other components in the composition may be used. Non-limiting examples of useful viscosity-modifying agents include hydrophobically modified alkali-soluble emulsion (HASE) polymers, ACULYN™ (Rohm and Haas Chemicals, Philadelphia, Pa.), nonionic polyurethane associative rheology modifiers, ACRYSOL™ (Rohm and Haas Chemicals, Philadelphia, Pa.), and cellulosic thickeners (e.g., hydroxypropyl cellulose, hydroxylmethyl cellulose, methyl cellulose, or carboxymethyl cellulose).

In certain preferred embodiments, the compositions of the invention comprise a solvent capable of dissolving the shape memory polymer. Non-limiting examples of such solvents include alcohols (e.g., ethanol, propylene glycol), ketones (e.g., acetone) and mineral oil. In some cases, it is advantageous to use a solvent with a sufficiently high vapor pressure such that the solvent has substantially evaporated after the composition has been applied to the hair.

Compositions of the present invention may also contain one or more additional ingredients conventionally incorporated into hair care compositions. Such additional ingredients include perfumes, dyes, buffering or pH adjusting agents, opacifiers, pearlizers, preservatives, antibacterial agents, antidandruff agents, vitamins, foam boosters, proteins, moisturizing agents, herb or other plant extracts, or other natural ingredients. The compositions of the present invention may include one or more additional components, such as one or more antimicrobials, antioxidants, buffering agents, chelating agents, colorants, conditioning agents, emollients, film formers, fragrances, humectants, lubricants, moisturizers, pigments, preservatives, stabilizers, or any combinations thereof.

Another aspect of the present invention is to provide a kit for styling hair. In its most general implementation, the kit includes components that contain or allow the user to prepare a composition comprising a shape memory polymer in an effective amount for styling hair. In certain preferred embodiments, the kit includes components that enable the user to prepare a thermoset shape memory polymer (e.g., a thermoset epoxy) by mixing together a thermoset resin and a hardener. For instance, the kit may include a first compartment containing a thermoset resin composition and a second compartment containing a hardener composition. In this implementation, the thermoset resin and hardener form a shape memory polymer when the compositions of the first and second compartments are mixed to form a hair styling composition. Optionally, the compositions in the kit may contain a cosmetically acceptable solvent or a viscosity-modifying agent, or both. When a viscosity modifying agent is present, it is preferable that the overall composition (e.g., after mixing) has a viscosity within the ranges defined herein.

### EXAMPLES

### Example 1

### Tress Shampoo Method

This example describes the procedure used to prepare hair tresses for experiments in which the percent curl retention is measured. The procedure that was used is as follows:
1. Wet hair tress under running water (95-105° F.) for 30 seconds.
2. Squeeze the tress lightly between middle and index fingers to remove excess water.
3. Measure out 0.5 ml shampoo and apply across top to bottom of the hair tress.
4. Use continuous downward massaging motion working shampoo into hair tress for 30 seconds.
5. Place hair tress under running water (95-105° F.) and use a downward messaging action to rinse for 30 seconds, or until all suds are removed.
6. Air dry hair tress.

### Example 2

### Hair Tress Treatment and Measurement Method For Curls

This example describes the procedure used to apply compositions containing shape memory polymers to hair tresses for experiments in which the percent curl retention is measured. The procedure that was used is as follows:
1. Apply specific amount of hair treatment solution (SMP) onto 0.25×8 inch light blonde hair tress (2.5 gm); distribute the solution evenly from root to tip and massage the solution into hair tress to cover all fibers.
2. Roll each tress on a 2-cm diameter wide, 6½ cm length roller and attach with 2 hair clips.
3. Provide stimulus for SMP curing, using curling iron or convection oven set at 120° F. (49° C.).
4. Cool tress to ambient conditions.
5. Carefully remove curled tresses from roller and record initial curl Lₚ (programmed length).
6. Straighten curls out with heat and pressure and measure the length Lₒ (creating temporary shape).
7. Apply an external stimulus for curl recovery and measure hair length vs. time (Lt). As discussed below, three different external stimuli were used: (a) a blow drier set to high (175° F.), warm water (100° F.), or an oven (120° F.).
8. Calculate % curl retention.

### Example 3

### Concentration Effect of TEMBO® on Hair Tress Curl Recovery

TEMBO® is a thermoset epoxy that is prepared by mixing a resin and a hardener (referred to as Part A and Part B) together. In this example, the two-part TEMBO® SMP system was combined in a 20 ml scintillation vial at ratio of 1:0.78 by weight Part A:Part B. Four samples were prepared by diluting the TEMBO® with 0, 1, 3, and 5 grams of acetone, resulting in samples having TEMBO® concentrations of 100%, 64%, 37%, and 26% respectively. The samples were mixed on a vortex mixer (Fisher Scientific) at the highest speed (10) for 30 seconds before application onto hair. One gram of each of the mixtures was applied onto separate light blonde hair (0.25×8 in) tresses, which were subsequently curled onto a roller.

To set the hair style, the hair was air dried for one hour and oven dried at 120° F. (49° C.) for 16 hrs. The rollers were then removed from oven and cooled to room temperature. Next, the hair tresses were removed from rollers and heated using a blow drier (Conair Pro Gold Bird) at the high setting for 30 seconds. The curls were straightened out with force and then the tresses were allowed to cool down to room temperature. The heat from the blow drier and straightening force created a straight hair style as the temporary shape. A blow drier was then used to recall the original curl in the tresses by applying heat at the high setting for one minute. This process resulted in significant curl recovery (>75%) for tresses treated with TEMBO® diluted with 0, 1, and 3 gm of acetone. For the samples diluted with 5 gm acetone, only ˜25-50% curl recovery was observed.

For the TEMBO® sample diluted with 3 gm of acetone, the temporary straight hair style was repeated using a blow drier, leading to similar curl recovery. This result indicates that the shape-recalling property of shape-memory polymers such as TEMBO® is durable. The temporary straight hair style was created again by running warm tap water (at 95-105° F. (i.e., 35 -41° C.)) over tress for 1 min, which resulted in a curl recovery of 25-50%.

### Example 4

### Solvent Effect on Hair Tress Curl Recovery Using TEMBO®

In this example, the solvent effect on hair tress curl recovery using TEMBO® was investigated. Three grams of the following solvents were combined in a 20 ml scintillation vial with 1.78 gm of TEMBO® (1:0.78 grams Part A:Part B) to form seven different samples: acetone, ethanol, phenoxyethanol, mineral oil, vegetable oil, propylene glycol, and water. Isododecane and cyclopentasiloxane were also tried, but because TEMBO® is insoluble in these two solvents, these mixtures were not tested. For each of the seven samples, the concentration of Tembo® was 37%, and 0.75 gm of each solvent/TEMBO® solution was applied onto the light blonde tresses. Using the tress treatment and measurement protocol described above in Examples 1 and 2, the hair tresses were placed rollers into oven set at 120° F. for 16 hrs. Lₚ and Lₒ were measured and used an oven set to 120° F. and 20% relative humidity to measure curl recovery over time. The results are provided in table below.

% Curl Recovery (Effect of Solvent)

| | Time (minutes) | | | | |
|---|---|---|---|---|---|
| Solvent | 0 | 1 | 2 | 5 | 15 |
| Acetone | 0 | 21 | 36 | 50 | 57 |
| Ethanol | 0 | 50 | 56 | 56 | 69 |
| Mineral Oil | 0 | 84 | 97 | 100 | — |
| Vegetable Oil | 0 | 0 | 0 | 0 | — |
| Phenoxyethanol | 0 | 0 | 0 | 0 | — |
| Propylene Glycol | 0 | 70 | 83 | 89 | — |

| | |
|---|---|
| Water | White precipitate formed in vial; Not tested |
| Isododecane | Tembo Insoluble; Not tested |
| cyclopentasiloxane (D5) | Tembo Insoluble; Not tested |

### Example 5

### Concentration Effect of TEMBO®/Mineral Oil Blend on Hair Tress Curl Recovery

In this example, the same protocol was carried out as in Example 4 except that 0.25 gm, 0.5 gm, 0.75 gm, and 1 gm of 37% TEMBO®/63% mineral oil was applied onto the light blonde tress. The rollers were placed in an oven set to 120° F. for 25 hrs instead of 16 hrs. The percent curl recovery is provided in the table below.

% Curl Recovery (37% Tembo ®/63% mineral oil)

| Concentration (gm)/ | Time (minutes) | | | | |
|---|---|---|---|---|---|
| Hair Tress | 0 | 1 | 2 | 5 | 15 |
| 0.25 | 0 | 9 | 14 | — | — |
| 0.5 | 0 | 12 | 29 | 38 | 59 |
| 0.75 | 0 | 70 | 74 | 78 | 83 |
| 1 | 0 | 59 | 70 | 76 | — |

The maximum curl recovery was observed for 0.75 gm application and seemed to decrease at 1 gm application. Furthermore, the performance decreased at 25 hrs of curing compared to 16 hrs as illustrated in Example 4.

### Example 6

### Effect of Residence Time in 120° F. Oven For Tresses Treated With TEMBO®

The effect of curing time in an oven set at 120° F. was measured for light blonde hair tresses treated with a 37% TEMBO®/63% mineral oil composition using the protocols described in Examples 1 and 2. As shown in the table below, greater than 50% curl recovery in 1 min was measured for curing times of 5, 16, 25, and 65 hrs. The best performance was seen for 16 hrs.

% Curl Recovery

| | Time (minutes) | | | | |
|---|---|---|---|---|---|
| Time (hrs) at 120 F. oven | 0 | 1 | 2 | 5 | 15 |
| 2 | 0 | 14 | 14 | — | — |
| 5 | 0 | 60 | 68 | 72 | — |
| 16 | 0 | 84 | 97 | 100 | — |
| 25 | 0 | 70 | 74 | 78 | 83 |
| 65 | 0 | 60 | 68 | 72 | — |

### Example 7

### Shape memory Polyurethane

Shape memory polyurethane was synthesized by dissolving PLA diol (10k) in toluene and heating to 75° C. for 30 min. Next, 1,6-hexamethylenediisocyanate (HDI) and 1,4-butanediol (BDO) were added into the reaction vessel at the molar ratio of PLA diol:HDI:BDO 1:2:1. The reaction mixture was stirred for 6 h. The polymer was isolated by dissolving the reaction mixture in chloroform followed by precipitation in ethanol. Shape memory polyurethanes exhibited Tₘ's from 140-200° C. and T_{g}'s from 50-60° C. Such shape memory polyurethanes may be used to set a permanent shape in hair by heating to a temperature above the polyurethane's Tₘ with a flat iron or curl iron. The permanent shape may then be recalled or set to a temporary shape with a blow drier.

### Example 8

### Shape Memory Polyamide-6 (nylon-6)

Shape memory polymers comprised of polyamide-6 (nylon-6) may be synthesized with Tₘ's in the range of of 215-223° C. and a T_{g} of ˜40° C. See, e.g., Angew. Chem. Int. Ed. 2002, 41: 2034-2057; Rompp Lexikon Chemie, Vol 5, Thieme, Stuttgart, 1998, p. 3415; Encyclopedia of Polymer Science and Engineering, Vol. 11, Wiley, NY, 1986, p. 366; Encyclopedia of Polymer Science and Engineering, Vol. 11, Wiley, NY, 1986, p. 349. Such shape memory polyurethanes may be used to set a permanent shape in hair by heating to a temperature above the polyurethane's Tₘ with a flat iron or curl iron. The permanent shape may then be recalled or set to a temporary shape with a blow drier.

The foregoing is illustrative of the present invention and is not to be construed as limiting. Although a few exemplary embodiments of this invention have been described, those skilled in the art will readily appreciate that many modifications are possible in the exemplary embodiments without materially departing from the novel teachings and advantages of this invention. Accordingly, it is to be understood that the foregoing is illustrative of the present invention and is not to be construed as limited to the specific embodiments disclosed, and that modifications to the disclosed embodiments, as well as other embodiments, are intended to be included within the scope of the appended claims.

1. A method for styling keratin fibers comprising the steps of:
(i) depositing a film of a thermoplastic polyurethane shape memory polymer (SMP) on a length of said keratin fibers, said SMP having a hard segment and a soft segment and a transition temperature (Tₜᵣₐₙₛ) in the range of about 120 to about 180° F., and being capable of forming cross-links at a temperature in the range of about 300 to about 500° F., and
(ii) styling said keratin fibers at a temperature in the range of about 300 to about 500° F. to cause said SMP to form cross-links

2. The method according to claim 1, wherein said SMP has a substantially crystalline domain which transitions to a substantially amorphous domain at said temperature Tₜᵣₐₙₛ.

3. The method according to claim 1, wherein said step of styling comprises curling said hair with a curling iron capable of heating the SMP to a temperature in the range of about 300 to about 400° F.

4. A method for recovering a desired hairstyle of a subject, the method comprising
applying a thermoplastic polyurethane shape memory polymer capable of forming cross-links to the hair of a subject, said shape memory polymer comprising a hard segment and a soft segment, wherein the soft segment has a temperature Tₜᵣₐₙₛ above which the soft segment melts or undergoes a glass transition;
styling the hair to a desired hairstyle;
setting the desired hairstyle by heating the hair to a setting temperature Tₛₑₜ which is greater than or equal to Tₜᵣₐₙₛ;
subsequently cooling the hair to a temperature below Tₜᵣₐₙₛ, and
recovering the desired hairstyle whenever it is lost by reheating the hair to a temperature greater than or equal to Tₜᵣₐₙₛ.

5. The method according to claim 4, wherein the step of setting the desired hairstyle comprises heating the hair to a setting temperature Tₛₑₜ in the range of about 300 to about 500° F.

6. The method according to claim 4, wherein the step of setting the desired hairstyle comprises heating the hair for from about 15 seconds to about an hour.

7. The method according to claim 6, wherein the step of setting the desired hairstyle comprises heating the hair for about 30 seconds in a temperature range of about 300 to about 500° F.

8. The method according to claim 4, wherein the step of setting the desired hairstyle is performed with a curling iron, flat iron, hair dryer, or curler dryer.

9. The method according to claim 4, wherein the step of recovering the desired hairstyle comprises heating the hair to a temperature Tₜᵣₐₙₛ in the range of about 120 to about 180° F.

10. The method according to claim 4, wherein the step of recovering the desired hairstyle comprises heating the hair for about 15 seconds to about 5 minutes.

11. The method according to claim 4, wherein the shape memory polymer is formed by chemical cross-linking.

12. The method according to claim 4, wherein the shape memory polymer is formed by physical cross-linking.

13. The method according to claim 4, wherein the desired hairstyle comprises at least one of curled hair or straight hair.

14. The method according to claim 13, wherein the percent curl recovery is greater than 50%.

15. The method according to claim 13, wherein the percent curl recovery is greater than 70%.
